(19) United States
(12) Patent Application Publication
(10) Pub. No.: US 2026/0061190 A1
(43) Pub. Date: Mar. 5, 2026

(54) SYSTEMS AND METHODS FOR ELECTROPORATION WITH CURRENT LIMITED ACTIVE ELECTRODE

(71) Applicant: St. Jude Medical, Cardiology Division, Inc., St. Paul, MN (US)

(72) Inventors: Lakshya Mittal, Dayton, MN (US); Derek C. Sutermeister, Ham Lake, MN (US); Gregory K. Olson, Elk River, MN (US); John Tranter, Minneapolis, MN (US); Isaac Remer, Richfield, MN (US); Lori Bailey, Chatfield, MN (US)

(21) Appl. No.: 19/291,093

(22) Filed: Aug. 5, 2025

Related U.S. Application Data
(60) Provisional application No. 63/679,876, filed on Aug. 6, 2024.

(51) Int. Cl. A61N 1/32; A61M 25/00; A61M 25/10; A61N 1/05; A61N 1/375

(52) U.S. Cl. CPC A61N 1/327; A61M 25/0009; A61M 25/0045; A61N 1/37516; A61M 25/10; A61N 1/05

(57) ABSTRACT

Systems and methods for electroporation are provided. An electroporation catheter includes an active electrode having a first surface area, and a return electrode having a second surface area, wherein the second surface area is greater than or equal to the first surface area to facilitate suppressing electric fields and/or preventing temperature rise at the return electrode when applying electrical energy between the active electrode and the return electrode to form a lesion proximate the active electrode.

### CROSS-REFERENCE TO RELATED APPLICATION

This application claims the benefit of, and priority to, U.S. Provisional Ser. No. 63/679,876, filed on Aug. 6, 2024, the entire content of which is hereby incorporated by reference herein.

### FIELD OF THE DISCLOSURE

The present disclosure relates generally to tissue ablation systems. In particular, the present disclosure is directed to electroporation systems with a current limited active electrode.

### BACKGROUND

It is generally known that ablation therapy may be used to treat various conditions afflicting the human anatomy. For example, ablation therapy may be used in the treatment of atrial arrhythmias or other arrhythmias (e.g., ventricular arrhythmias). Further, ablation therapy may be used for ablating soft tissue, cancerous tissue, benign tumors, etc. When tissue is ablated, or at least subjected to ablative energy generated by an ablation generator and delivered by an ablation catheter, lesions form in the tissue. Electrodes mounted on or in ablation catheters are used to cause tissue destruction in cardiac tissue to correct conditions such as ventricular and atrial arrhythmias (including, but not limited to, ectopic atrial tachycardia, atrial fibrillation, and atrial flutter).

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is an ablation technique that involves applying strong electric fields that induce pore formation in the cellular membrane. The electric field may be induced by applying a relatively short duration pulse which may last, for instance, from a nanosecond to several milliseconds and generate a moderate amount of heating. Such a pulse may be repeated to form a pulse train. When such an electric field is applied to tissue in an in vivo setting, the cells in the tissue are subjected to an increased trans-membrane potential, which opens the pores on the cell plasma membrane. Electroporation may be reversible (i.e., the temporally-opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction, for instance by causing irreversible electroporation.

For example, pulsed field ablation (PFA) may be used to perform instantaneous pulmonary vein isolation (PVI). PFA generally involves delivering high voltage pulses from electrodes disposed on a catheter. For example, in at least some systems, voltage pulses may range from less than about 50 volts to about 10,000 volts or higher. These fields may be applied between pairs of electrodes (bipolar therapy) or between one or more electrodes and a return patch (monopolar therapy).

In PFA, different waveforms may be used to achieve different goals. For example, some waveforms may result in larger or smaller lesion size than other waveforms. Further, some waveforms result in higher or lower overall energy delivery than other waveforms (less overall energy delivery generally corresponds to less effected volume/cell destruction and less heating of the target tissue). As another example, some waveforms are more likely to induce muscular contractions in a patient. Generally, it is desirable to minimize thermal heating of the tissue, and to have little to no skeletal muscle recruitment (i.e., avoiding muscle contractions). In addition, it is also generally desirable to reduce the likelihood of waveforms generating sustained atrial arrhythmias.

During application of PFA, electrode geometry (e.g., electrode shape, length, interelectrode distances, etc.) determines the size of the electric field generated, and accordingly, the lesion size. In at least some known systems, a return electrode is sized (relative to an active electrode) such that the return electrode is current limiting (as opposed to the active electrode). This may result in undesirable heating and shadow lesion formation at the return electrode.

### BRIEF SUMMARY OF THE DISCLOSURE

In one aspect, an electroporation catheter is provided. The electroporation catheter includes an active electrode having a first surface area, and a return electrode having a second surface area, wherein the second surface area is greater than or equal to the first surface area to facilitate suppressing electric fields and/or preventing temperature rise at the return electrode when applying electrical energy between the active electrode and the return electrode to form a lesion proximate the active electrode.

In another aspect, an electroporation system is provided. The electroporation system includes a pulse generator, and an electroporation catheter coupled to the pulse generator. The electroporation catheter includes an active electrode having a first surface area, and a return electrode having a second surface area, wherein the second surface area is greater than or equal to the first surface area to facilitate suppressing electric fields and/or preventing temperature rise at the return electrode when applying, using the pulse generator, electrical energy between the active electrode and the return electrode to form a lesion proximate the active electrode.

In yet another aspect, a method of assembling an electroporation catheter is provided. The method includes positioning an active electrode on a shaft that extends along a longitudinal axis, the active electrode having a first surface area, and positioning a return electrode on the shaft, the second electrode having a second surface area that is greater than or equal to the first surface area to facilitate suppressing electric fields and/or preventing temperature rise at the return electrode when applying electrical energy between the active electrode and the return electrode to form a lesion proximate the active electrode.

The active electrode may include a plurality of active electrodes, the return electrode may include a plurality of return electrodes, and a sum of the second surface areas of the plurality of return electrodes may be greater than a sum of the first surface areas of the plurality of active electrodes.

The electroporation catheter may be a linear catheter including a shaft extending along a longitudinal axis. The active electrode and the return electrode may be disposed on the shaft. The active electrode may include a tip electrode located at a distal end of the shaft and the return electrode may be proximal of the tip electrode.

The active electrode may have a first length, and the return electrode may have a second length that is greater than or equal to the first length. The active electrode may have a first length in a direction parallel to the longitudinal axis, the return electrode may include a second length in the direction parallel to the longitudinal axis that is greater than or equal to the first length.

The active electrode may include a first diameter, and the return electrode may include a second diameter that is greater than or equal to the first diameter. The active electrode may have a first diameter in a direction perpendicular to the longitudinal axis, and the return electrode may have a second diameter in a direction perpendicular to the longitudinal axis that is greater than or equal to the first diameter.

At least one of the active electrode and the return electrode may include a plurality of independently activatable sub-electrodes. Each of the plurality of sub-electrodes may include a flex circuit.

The electroporation catheter may include an irrigation architecture configured to deliver a first irrigant to the active electrode and deliver a second irrigant to the return electrode. The first irrigant may have a lower conductivity than the second irrigant. The first irrigant may include a cytotoxic agent.

At least one of the active electrode and the return electrode may include one of a conductive balloon and a conductive mesh.

The second surface area may be greater than or equal to the first surface area to facilitate reducing the likelihood of at least one of lesion, char, blood coagulation, and hemolysis.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a schematic and block diagram view of a system for electroporation therapy.

FIG. 2 is a schematic diagram of one embodiment of a catheter assembly that may be used with the system shown in FIG. 1.

FIG. 3 is a schematic diagram of one embodiment of a catheter assembly that may be used with the system shown in FIG. 1.

FIG. 4 is a schematic diagram of one embodiment of a catheter assembly that may be used with the system shown in FIG. 1.

FIG. 5 is a schematic diagram of one embodiment of a catheter assembly that may be used with the system shown in FIG. 1.

FIG. 6 is a schematic diagram of one embodiment of a catheter assembly that may be used with the system shown in FIG. 1.

FIG. 7 is a schematic diagram of one embodiment of a catheter assembly that may be used with the system shown in FIG. 1.

### DETAILED DESCRIPTION OF THE DISCLOSURE

The present disclosure provides systems and methods for electroporation. An electroporation catheter includes an active electrode having a first surface area, and a return electrode having a second surface area, wherein the second surface area is greater than or equal to the first surface area to facilitate suppressing electric fields and/or preventing temperature rise at the return electrode when applying electrical energy between the active electrode and the return electrode to form a lesion proximate the active electrode.

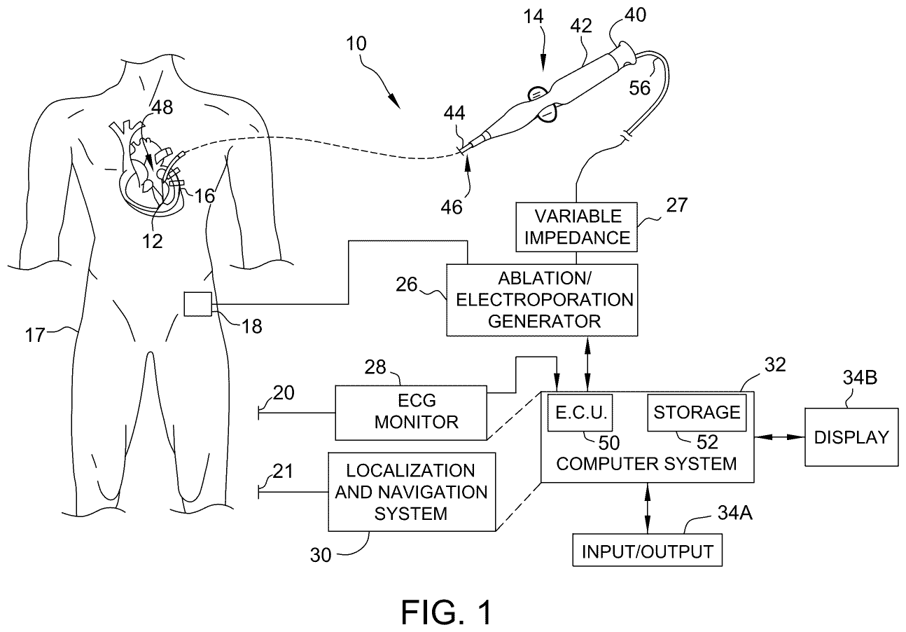

FIG. 1 is a schematic and block diagram view of a system 10 for electroporation therapy. In general, system 10 includes a catheter electrode assembly 12 disposed at a distal end 48 of a catheter 14. As used herein, “proximal” refers to a direction toward the end of the catheter near the clinician and “distal” refers to a direction away from the clinician and (generally) inside the body of a patient. The electrode assembly includes one or more individual, electrically-isolated electrode elements. Each electrode element, also referred to herein as a catheter electrode, is individually wired such that it can be selectively paired or combined with any other electrode element to act as a bipolar or a multi-polar electrode.

System 10 may be used for irreversible electroporation (IRE) to destroy tissue. In particular, system 10 may be used for electroporation-induced therapy that includes delivering electrical pulses in such a manner as to directly cause an irreversible loss of plasma membrane integrity leading to its breakdown and cell destruction. This mechanism of cell destruction may be viewed as an “outside-in” process, meaning that the disruption of the outside plasma membrane of the cell causes detrimental effects to the inside of the cell. Sometimes these electrical pulses may directly manipulate and damage the intracellular organelles to induce cell death, without causing a significant amount of damage to the cell membrane. Typically, for classical electroporation, electric energy may be delivered as a pulsed electric field in the form of short-duration pulses (e.g., having a 10 nanosecond (ns) to 100 millisecond (ms) duration) between closely spaced electrodes capable of delivering an electric field strength of about 0.05 to 100.0 kilovolts/centimeter (kV/cm). System 10 may be used for high output (e.g., high voltage and/or high current) electroporation procedures (and may also be used for low output electroporation procedures, as described herein). Further, system 10 may be used with a linear catheter, a loop catheter, and/or a basket catheter. In some embodiments, system 10 is used for reversible electroporation instead of or in addition to irreversible electroporation. For example, system 10 may be used to deliver drugs and/or molecules to cells.

In one embodiment, stimulation is delivered selectively (e.g., between pairs of electrodes) on catheter 14. Further, the electrodes on catheter 14 may be switchable between being connected to a 3D mapping system and being connected to an electroporation generator.

Irreversible electroporation through a multi-electrode catheter may enable pulmonary vein isolation in as few as one shock per vein, which may produce much shorter procedure times compared to sequentially positioning a radiofrequency (RF) ablation tip around a vein. Further, irreversible electroporation may be used for focal ablation procedures to create lines and/or to ablate focal spots. Notably, the embodiments described herein may be used with any suitable irreversible electroporation application.

It should be understood that while the energization strategies are described as involving square wave pulses, embodiments may use variations and remain within the spirit and scope of the disclosure. For example, triangular pulses, sinusoidal pulses, exponentially-decaying pulses, exponentially-increasing pulses, and combinations may be used.

Further, it should be understood that the mechanism of cell destruction in electroporation is not primarily due to heating effects, but rather to cell membrane disruption (e.g., through pore formation and/or other cell membrane damage, such as lipid peroxidation), damage to intracellular organelles and oxidate stress etc., causing a cell wide effect through application of a high-voltage electric field. Thus, electroporation may avoid some possible thermal effects that may occur when using radio frequency (RF) energy.

With this background, and now referring again to FIG. 1, system 10 includes a catheter electrode assembly 12 including at least one catheter electrode.

Electrode assembly 12 is incorporated as part of a medical device such as a catheter 14 for electroporation therapy of tissue 16 in a body 17 of a patient. In the illustrative embodiment, tissue 16 includes heart or cardiac tissue. It should be understood, however, that embodiments may be used to conduct electroporation therapy with respect to a variety of other body tissues (e.g., renal tissue, tumors, etc.).

FIG. 1 further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode, and may include split patch electrodes (as described herein). Further, in some embodiments, in a multiplexing arrangement, therapy may repeatedly switch between using a different return electrodes 18, 20, 21 or multiple in combination. In other embodiments, return electrodes 18, 20, and 21 may be any other type of electrode suitable for use as a return electrode including, for example, one or more catheter electrodes or both patch and catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with an electroporation energization strategy, which may be predetermined or may be user-selectable. For electroporation therapy, generator 26 may be configured to produce an electric energy that is delivered via electrode assembly 12 as a pulsed electric field in the form of short-duration square wave pulses (e.g., a nanosecond to several milliseconds duration, or any duration suitable for electroporation) between closely spaced electrodes capable of delivering an electric field strength (i.e., at the tissue site) of about 0.05 to 100.0 kV/cm. The amplitude and pulse width needed for irreversible electroporation are nearly inversely related. That is, as pulse widths are decreased, the amplitude may generally be increased to achieve chronaxie. The electric energy may be delivered, for example, using a fixed voltage delivery system (in which a fixed voltage is applied, independent of a patient impedance) or using a fixed current delivery system (in which a fixed current is achieved by adjusting the voltage based on the patient impedance). In a fixed current delivery system, the patient impedance may be determined, for example, by delivering a relatively small voltage pulse and measuring current to calculate impedance, or by delivering an AC current waveform and measuring voltage to calculate impedance. A fixed current system may also involve measuring current (e.g., before or during therapy delivery) and adjusting voltage accordingly. For example, current may be measured during delivery of a first therapy pulse (or during a pre-therapy pulse with a relatively low voltage), an impedance may be calculated from the measured current, and the voltage may be adjusted (and then left unchanged) to obtain the desired current during therapy. In another example, current may be measured during one or more pulses delivered during therapy, the impedance may be calculated for each pulse that the current was measured for, and the voltage of each subsequent pulse may be actively adjusted.

Electroporation generator 26, sometimes also referred to herein as a DC energy source, is a biphasic electroporation generator 26 configured to generate a series of energy pulses that all produce current in two directions (i.e., positive and negative pulses). In other embodiments, electroporation generator is a monophasic or polyphasic electroporation generator. In some embodiments, electroporation generator 26 is configured to output energy in pulses at selectable energy levels, such as fifty joules, one hundred joules, two hundred joules, and the like. Other embodiments may have more, or fewer energy settings and the values of the available setting may be the same or different (settings may include, e.g., waveform parameters, voltage, current, number of applications, etc.). For successful electroporation, some embodiments utilize the two hundred joule output level. For example, electroporation generator 26 may output a pulse having a peak magnitude from about 10 Volts (V) to about 20,000 V. Other embodiments may output any other suitable positive or negative voltage.

In some embodiments, a variable impedance 27 allows the impedance of system 10 to be varied to limit arcing. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

With continued reference to FIG. 1, as noted above, catheter 14 may include functionality for electroporation and in certain embodiments also additional ablation functions (e.g., RF ablation). It should be understood, however, that in those embodiments, variations are possible as to the type of ablation energy provided (e.g., cryoablation, ultrasound, etc.).

In the illustrative embodiment, catheter 14 includes a cable connector or interface 40, a handle 42, and a shaft 44 having a proximal end 46 and a distal end 48. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter, and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, biologics, and/or surgical tools or instruments. Shaft 44 may be made from conventional materials such as polyurethane and defines one or more lumens configured to house and/or transport electrical conductors, fluids or surgical tools, as described herein. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

Localization and navigation system 30 may be provided for visualization, mapping and navigation of internal body structures. Localization and navigation system 30 may include conventional apparatus known generally in the art. For example, localization and navigation system 30 may be substantially similar to the EnSite Precision™ System, commercially available from Abbott Laboratories, and as generally shown in commonly assigned U.S. Pat. No. 7,263,397 titled “Method and Apparatus for Catheter Navigation and Location and Mapping in the Heart”, the entire disclosure of which is incorporated herein by reference. In another example, localization and navigation system 30 may be substantially similar to the EnSite X™ System, as generally shown in U.S. Pat. App. Pub. No. 2020/0138334 titled “Method for Medical Device Localization Based on Magnetic and Impedance Sensors”, the entire disclosure of which is incorporated herein by reference. It should be understood, however, that localization and navigation system 30 is an example only, and is not limiting in nature. Other technologies for locating/navigating a catheter in space (and for visualization) are known, including for example, the CARTO® navigation and location system of Biosense Webster, Inc., the Rhythmia® system of Boston Scientific Scimed, Inc., the KODEX® system of Koninklijke Philips N.V., the AURORA® system of Northern Digital Inc., commonly available fluoroscopy systems, or a magnetic location system such as the gMPS system from Mediguide Ltd.

In this regard, some of the localization, navigation and/or visualization systems may include a sensor for producing signals indicative of catheter location information, and may include, for example, one or more electrodes in the case of an impedance-based localization system, or alternatively, one or more coils (i.e., wire windings) configured to detect one or more characteristics of a magnetic field, for example in the case of a magnetic-field based localization system. As yet another example, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety.

Pulsed field ablation (PFA), which is a methodology for achieving irreversible electroporation and cell death, may be implemented using the systems and methods described herein. In some cases, PFA may be used at specific cardiac tissue sites such as the pulmonary veins to perform a pulmonary vein isolation (PVI), or to perform focal ablation. In PFA, electric fields may be applied between catheter electrodes (in a bipolar approach) or between one or more catheter electrodes and a return patch (in a monopolar approach) or a combination of catheter electrodes and a return patch (in a multipolar approach). There are advantages and disadvantages to each of these approaches.

Both approaches, using an appropriate electrode geometry, are able to provide contiguous lesions. For lesion size and proximity, the monopolar approach can potentially create deeper lesions with the same applied voltage. Further, the monopolar approach may be able to create lesions from a distance (e.g., generally proximate, but not necessarily contacting tissue). The bipolar approach may create smaller lesions, requiring closer proximity or contact with tissue to create transmural lesions (depending on, for example, tissue thickness). To monitor operation of system 10, one or more impedances between catheter electrodes 144 and/or return electrodes 18, 20, and 21 may be measured. For example, for system 10, impedances may be measured as described in U.S. Patent Application Publication No. 2019/0117113, filed on Oct. 23, 2018, U.S. Patent Application Publication No. 2019/0183378, filed on Dec. 19, 2018, and U.S. Patent Application Publication No. 2023/0190364, filed on Nov. 18, 2022, all of which are incorporated by reference herein in their entirety.

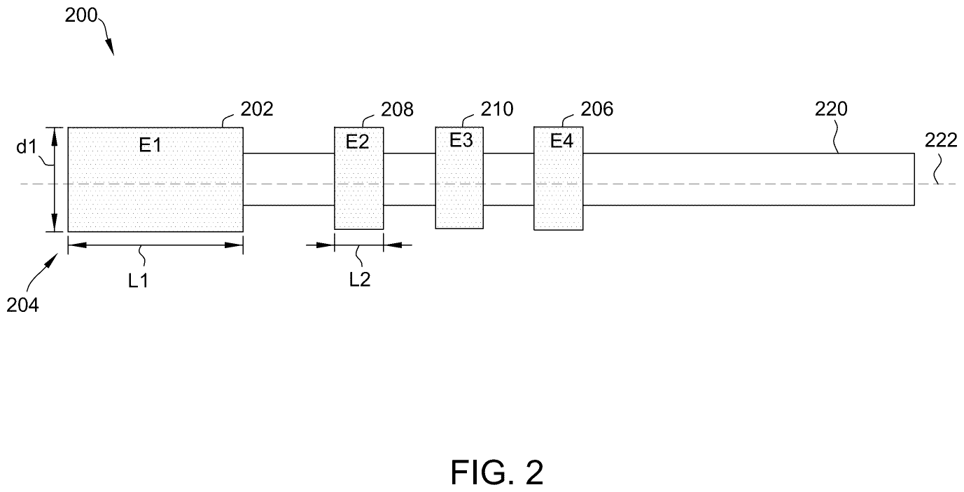

FIG. 2 is a schematic diagram of one embodiment of a catheter assembly 200 that may be used with catheter 14 in system 10. Catheter assembly 200 may be referred to as a linear catheter. Although the systems and methods disclosed herein are described in the context of linear catheters, those of skill in the art will appreciate that the techniques discussed herein may be implemented in other suitable catheter geometries as well.

As shown in FIG. 2, catheter assembly 200 includes a tip electrode 202 (E1) at a distal end 204 of catheter assembly 200. Catheter assembly 200 further includes a proximal electrode 206 (E4), a first intermediate electrode 208 (E2), and a second intermediate electrode 210 (E3). Tip electrode 202, proximal electrode 206, first intermediate electrode 208, and second intermediate electrode 210 are disposed along a shaft 220 that extends along a longitudinal axis 222.

Tip electrode 202 may be, for example, a dome-shaped electrode. Further, proximal electrode 206, first intermediate electrode 208, and second intermediate electrode 210 may be, for example ring electrodes. Alternatively, tip electrode 202, proximal electrode 206, first intermediate electrode 208, and second intermediate electrode 210 may each be any suitable type and/or shape of electrode.

In this embodiment, tip electrode 202 has a diameter, d1, and a length L1, and proximal electrode 206, first intermediate electrode 208, and second intermediate electrode 210 each have a diameter, d1, and a length L2 (where L2 is less than L1). Accordingly, tip electrode 202 is relatively large (and has a larger surface area) compared to each of proximal electrode 206, first intermediate electrode 208, and second intermediate electrode 210. In some embodiments, the diameter, d1, is measured in a direction perpendicular to the longitudinal axis 222 and the length, L1, is measured in a direction parallel to the longitudinal axis 222, although the diameter, d1, and the length, L1, may be measured in any orientation relative to each other and/or the longitudinal axis 222 without departing from the scope of the disclosure.

For PFA, lesion formation depends upon waveform parameters (e.g., pulse width, voltage, number of pulses, pulse period, etc.) and electrode geometry.

When electroporation pulses are applied between the larger tip electrode 202 and a smaller return electrode (e.g., proximal electrode 206, first intermediate electrode 208, or second intermediate electrode 210), the smaller return electrode has a relatively high resistance (due to its smaller size) that limits current from tip electrode 202. Accordingly, the smaller return electrode, not tip electrode 202, is the current limiting electrode. Further, the smaller return electrode also results in a relatively high current density (due to its smaller size) that may lead to higher electric fields (and corresponding higher electric field gradients), as well as significantly higher levels of temperature rise at smaller current levels, as compared to tip electrode 202. Due to its larger surface area, tip electrode 202 generates lower electric fields than the smaller return electrode and does not heat up as much as the smaller return electrode when carrying the same current due to reduced current density.

These phenomena may create challenges when applying higher voltages and currents to create deeper lesions, as the limited current, higher electric fields, and increased temperatures at the return electrode are generally undesirable. For example, the higher electric field (and electric field gradient) at the return electrode (as compared to tip electrode 202) may create a shadow lesion at the return electrode (which is undesirable). Accordingly, the systems and methods described herein facilitate addressing these potential issues.

More specifically, the systems and methods described herein improve lesion formation by using low resistance return electrodes that cause the active electrode to be the current limiting electrode (e.g., a tip electrode, such as tip electrode 202). This involves using an active electrode sized to accommodate lesion requirements, and using return electrodes that avoid limiting current flow. In at least some of the embodiments described herein, this is achieved by using one or more return electrodes that have a surface area (e.g., a sum of the surface area of the one or more return electrodes) greater than or equal to a surface area of the active electrode or, in some embodiments, a sum of the surface area of a plurality of active electrodes. This reduces the current density and electric field (and electric field gradient) at the one or more return electrodes.

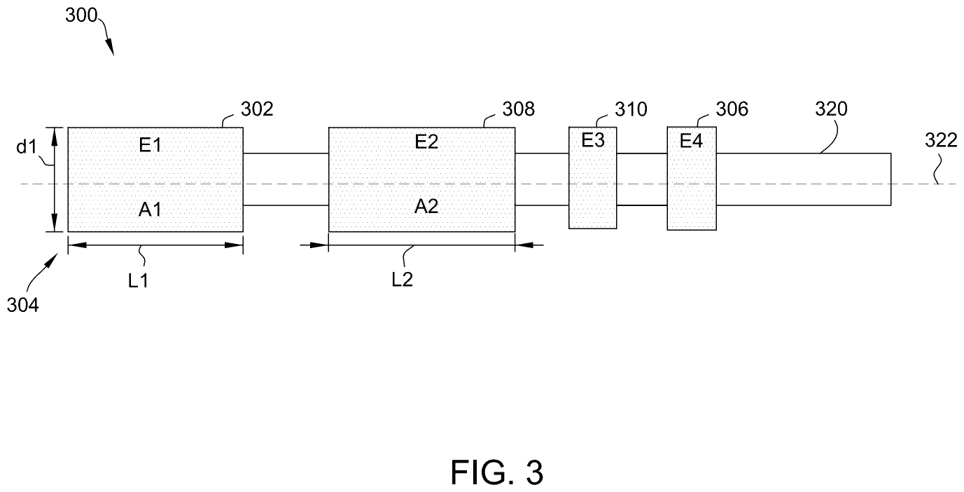

For example, FIG. 3 is a schematic diagram of one embodiment of a catheter assembly 300 that may be used with catheter 14 in system 10. Catheter assembly 300 includes a tip electrode 302 (E1) at a distal end 304 of catheter assembly 300. Catheter assembly 300 further includes a proximal electrode 306 (E4), a first intermediate electrode 308 (E2), and a second intermediate electrode 310 (E3). Tip electrode 302, proximal electrode 306, first intermediate electrode 308, and second intermediate electrode 310 are disposed along a shaft 320 that extends along a longitudinal axis 322.

Tip electrode 302 may be, for example, a dome-shaped electrode. Using a dome-shaped electrode as tip electrode 302 facilitates reducing edge effects, reducing the likelihood of blood coagulation and/or hemolysis. In general, smaller electrodes and/or sharper electrode edges may facilitate relatively high current density and electric field generation, possibly increasing the likelihood of heating, blood coagulation, and/or hemolysis. Further, proximal electrode 306, first intermediate electrode 308, and second intermediate electrode 310 may be, for example ring electrodes. Alternatively, tip electrode 302, proximal electrode 306, first intermediate electrode 308, and second intermediate electrode 310 may each be any suitable type and/or shape of electrode.

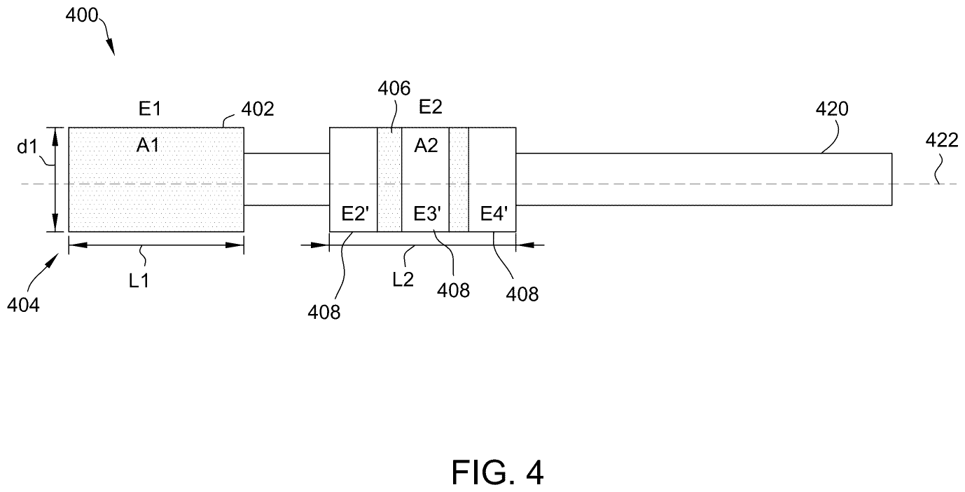

For example, in some embodiments, at least one of tip electrode 302, proximal electrode 306, first intermediate electrode 308, and second intermediate electrode 310 may be implemented using a selectively inflatable conductive balloon or a selectively expandable conductive mesh (e.g., expandable into a spherical shape or a toroidal shape). Using a conductive balloon or conductive mesh facilitates increasing a surface area of one or more of tip electrode 302, proximal electrode 306, first intermediate electrode 308, and second intermediate electrode 310. Further, those of skill in the art will appreciate that a conductive balloon and/or a conductive mesh may be implemented as electrodes in the other embodiments described herein (e.g., the embodiments shown and discussed below in association with FIGS. 4-7).

In this embodiment, tip electrode 302 has a diameter, d1, and a length L1, and first intermediate electrode 308 has a diameter, d1, and a length L2. Here, unlike catheter assembly 200 (shown in FIG. 2), L2 is greater than or equal to L1. Accordingly, first intermediate electrode 308 has a surface area (A2) that is greater than or equal to a surface area (A1) of tip electrode 302. In some embodiments, the diameter, d1, is measured in a direction perpendicular to the longitudinal axis 322 and the length, L1, is measured in a direction parallel to the longitudinal axis 322, although the diameter, d1, and the length, L1, may be measured in any orientation relative to each other and/or the longitudinal axis 322 without departing from the scope of the disclosure.

When using tip electrode 302 as the active electrode and first intermediate electrode 308 as the return electrode, first intermediate electrode 308 has a lower resistance and a lower current density than a smaller return electrode would. Accordingly, first intermediate electrode 308 generates lower electric fields (and electric field gradients) and reduces the possibility of generating a shadow lesion at first intermediate electrode 308 (as compared to a smaller return electrode). Further, first intermediate electrode 308 experiences less of a temperature rise (as compared to a smaller return electrode), and reduces the possibility of generating char, blood coagulation, and hemolysis at first intermediate electrode 308 (as compared to a smaller return electrode).

In the embodiment shown in FIG. 3, proximal electrode 306 and second intermediate electrode 310 are shown as having a smaller surface area than tip electrode 302. However, those of skill in the art will appreciate that proximal electrode 306 and/or second intermediate electrode 310 may, in some embodiments, in addition to or alternatively to first intermediate electrode 308, have a surface area greater than or equal to a surface area of tip electrode 302. In such embodiments, proximal electrode 306 and/or second intermediate electrode 310 may be used as a return electrode, realizing the same advantages as those described above with respect to first intermediate electrode 308. Further, in some embodiments, proximal electrode 306, first intermediate electrode 308, and second intermediate electrode 310 may be activated together to function as single, larger effective return electrode (resulting in lower electric fields and less temperature rise at the effective return electrode).

FIG. 4 is a schematic diagram of another embodiment of a catheter assembly 400 that may be used with catheter 14 in system 10. Catheter assembly 400 includes a tip electrode 402 (E1) at a distal end 404 of catheter assembly 400.

Catheter assembly 400 further includes a proximal electrode 406 (E2). Tip electrode 402 and proximal electrode 406 are disposed along a shaft 420 that extends along a longitudinal axis 422.

Tip electrode 402 may be, for example, a dome-shaped electrode. Using a dome-shaped electrode as tip electrode 402 facilitates reducing edge effects, reducing the likelihood of blood coagulation and/or hemolysis. In general, smaller electrodes and/or sharper electrode edges may facilitate relatively high current density and electric field generation, possibly increasing the likelihood of heating, blood coagulation, and/or hemolysis. Alternatively, tip electrode 402 may be any suitable type and/or shape of electrode.

In this embodiment, proximal electrode 406 includes a plurality of sub-electrodes 408 (E2′, E3′, E4′). Each sub-electrode 408 may be, for example, a flex circuit printed on proximal electrode 406. Alternatively, proximal electrode 406 and sub-electrodes 408 may be any suitable type and/or shape of electrode. In one non-limiting embodiment, one or more of the plurality of sub-electrodes 408 may be printed on a substrate (not labeled), which in some embodiments, may be an insulator.

For PFA applications, sub-electrodes 408 may be activated together (e.g., in a “ganged” configuration) to function as a single, larger effective electrode (i.e., proximal electrode 406). In contrast, for mapping and/or recording applications, sub-electrodes 408 may be activated independently from one another (e.g., in an “unganged” configuration).

In this embodiment, tip electrode 402 has a diameter, d1, and a length L1, and proximal electrode 406 has a diameter, d1, and a length L2. Here, unlike catheter assembly 200 (shown in FIG. 2), L2 is greater than or equal to L1. Accordingly, proximal electrode 406 (including sub-electrodes 408) has a surface area (A2) that is greater than or equal to a surface area (A1) of tip electrode 402. In some embodiments, the diameter, d1, is measured in a direction perpendicular to the longitudinal axis 422 and the length, L1, is measured in a direction parallel to the longitudinal axis 422, although the diameter, d1, and the length, L1, may be measured in any orientation relative to each other and/or the longitudinal axis 422 without departing from the scope of the disclosure.

When using tip electrode 402 as the active electrode and proximal electrode 406 as the return electrode, proximal electrode 406 has a lower resistance and a lower current density than a smaller return electrode would. Accordingly, proximal electrode 406 generates lower electric fields (and electric field gradients) and reduces the possibility of generating a shadow lesion at proximal electrode 406 (as compared to a smaller return electrode). Further, proximal electrode 406 experiences less of a temperature rise (as compared to a smaller return electrode), and reduces the possibility of generating char, blood coagulation, and hemolysis at proximal electrode 406 (as compared to a smaller return electrode).

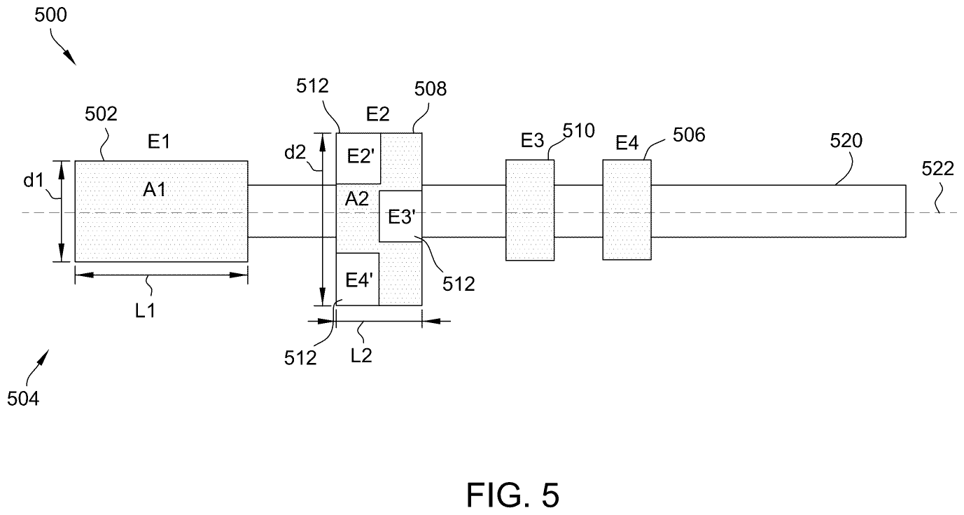

FIG. 5 is a schematic diagram of another embodiment of a catheter assembly 500 that may be used with catheter 14 in system 10. Catheter assembly 500 includes a tip electrode 502 (E1) at a distal end 504 of catheter assembly 500.

Catheter assembly 500 further includes a proximal electrode 506 (E4), a first intermediate electrode 508 (E2), and a second intermediate electrode 510 (E3). Tip electrode 502, proximal electrode 506, first intermediate electrode 508, and second intermediate electrode 510 are disposed along a shaft 520 that extends along a longitudinal axis 522.

Tip electrode 502 may be, for example, a dome-shaped electrode. Using a dome-shaped electrode as tip electrode 502 facilitates reducing edge effects, reducing the likelihood of blood coagulation and/or hemolysis. In general, smaller electrodes and/or sharper electrode edges may facilitate relatively high current density and electric field generation, possibly increasing the likelihood of heating, blood coagulation, and/or hemolysis. Alternatively, tip electrode 502 may be any suitable type and/or shape of electrode.

In this embodiment, first intermediate electrode 508 includes a plurality of sub-electrodes 512 (E2′, E3′, E4′). Each sub-electrode 512 may be, for example, a flex circuit printed on first intermediate electrode 508. Further, proximal electrode 506 and second intermediate electrode 510 may be ring electrodes. Alternatively, first intermediate electrode 508, sub-electrodes 512, proximal electrode 506, and second intermediate electrode 510 may each be any suitable type and/or shape of electrode. In one non-limiting embodiment, one or more of the plurality of sub-electrodes 512 may be printed on a substrate (not labeled), which in some embodiments, may be an insulator.

For PFA applications, sub-electrodes 512 may be activated together (e.g., in a “ganged” configuration) to function as a single, larger effective electrode (i.e., first intermediate electrode 508). In contrast, for mapping and/or recording applications, sub-electrodes 512 may be activated independently from one another (e.g., in an “unganged” configuration).

In this embodiment, tip electrode 502 has a diameter, d1, and a length L1, and first intermediate electrode 508 has a diameter, d2, and a length L2. Here, although L2 is less than L1, d2 is greater than d1, such that first intermediate electrode 508 (including sub-electrodes 512) has a surface area (A2) that is greater than or equal to a surface area (A1) of tip electrode 502. In some embodiments, the diameter, d1, is measured in a direction perpendicular to the longitudinal axis 522 and the length, L1, is measured in a direction parallel to the longitudinal axis 522, although the diameter, d1, and the length, L1, may be measured in any orientation relative to each other and/or the longitudinal axis 522 without departing from the scope of the disclosure.

When using tip electrode 502 as the active electrode and first intermediate electrode 508 as the return electrode, first intermediate electrode 508 has a lower resistance and a lower current density than a smaller return electrode would. Accordingly, first intermediate electrode 508 generates lower electric fields (and electric field gradients) and reduces the possibility of generating a shadow lesion at first intermediate electrode 508 (as compared to a smaller return electrode). Further, first intermediate electrode 508 experiences less of a temperature rise (as compared to a smaller return electrode), and reduces the possibility of generating char, blood coagulation, and hemolysis at first intermediate electrode 508 (as compared to a smaller return electrode).

Further, having first intermediate electrode 508 wider and shorter than tip electrode 502 (i.e., d2>d1, L2<L1) enables forming relatively uniform lesions regardless of an angle between catheter assembly 500 and tissue. Further, this enables forming wider lesions using a linear catheter than may be possible using alternative electrode configurations on a linear catheter.

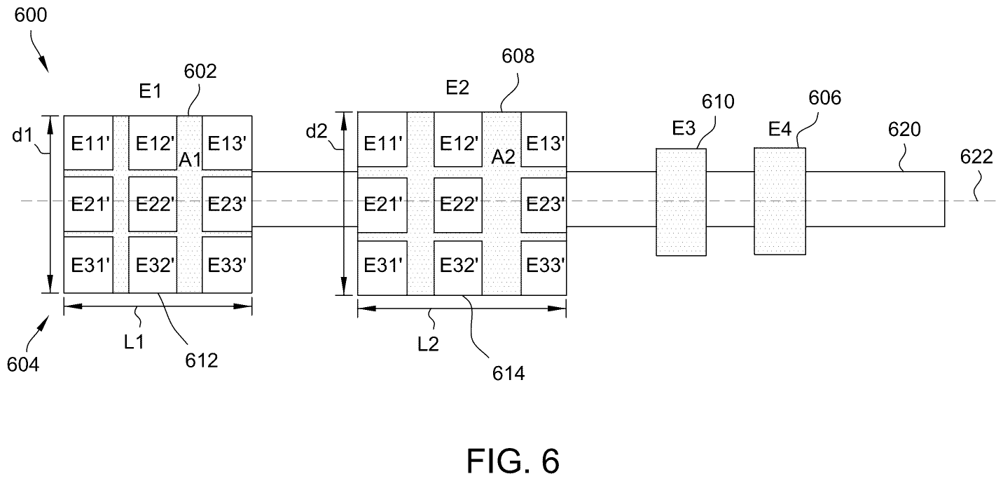

FIG. 6 is a schematic diagram of another embodiment of a catheter assembly 600 that may be used with catheter 14 in system 10. Catheter assembly 600 includes a tip electrode 602 (E1) at a distal end 604 of catheter assembly 600. Catheter assembly 600 further includes a proximal electrode 606 (E4), a first intermediate electrode 608 (E2), and a second intermediate electrode 610 (E3). Tip electrode 602, proximal electrode 606, first intermediate electrode 608, and second intermediate electrode 610 are disposed along a shaft 620 that extends along a longitudinal axis 622.

Tip electrode 602 may be, for example, a dome-shaped electrode. Using a dome-shaped electrode as tip electrode 602 facilitates reducing edge effects, reducing the likelihood of blood coagulation and/or hemolysis. In general, smaller electrodes and/or sharper electrode edges may facilitate relatively high current density and electric field generation, possibly increasing the likelihood of heating, blood coagulation, and/or hemolysis. Alternatively, tip electrode 602 may be any suitable type and/or shape of electrode.

Further, in this embodiment, tip electrode 602 includes a plurality of first sub-electrodes 612 (E11′, E12′, E13′, E21′, E22′, E23′, E31′, E32′, and E33′). Each first sub-electrode 612 may be, for example, a flex circuit printed on tip electrode 602. In addition, first intermediate electrode 608 includes a plurality of second sub-electrodes 614 (E11′, E12′, E13′, E21′, E22′, E23′, E31′, E32′, and E33′). Each second sub-electrode 614 may be, for example, a flex circuit printed on first intermediate electrode 608. Further, proximal electrode 606 and second intermediate electrode 610 may be ring electrodes. Alternatively, tip electrode 602, first sub-electrodes 612, first intermediate electrode 608, second sub-electrodes 614, proximal electrode 606, and second intermediate electrode 610 may each be any suitable type and/or shape of electrode. In one non-limiting embodiment, one or more of the plurality of first sub-electrodes 612 and the plurality of second sub-electrodes 614 may be printed on a substrate (not labeled), which in some embodiments, may be an insulator.

For PFA applications, first sub-electrodes 612 may be activated together (e.g., in a “ganged” configuration) to function as a single, larger effective electrode (i.e., tip electrode 602). In contrast, for mapping and/or recording applications, first sub-electrodes 612 may be activated independently from one another (e.g., in an “unganged” configuration). Similarly, second sub-electrodes 614 may be activated together for PFA applications and activated independently from one another for mapping and/or recording applications.

In this embodiment, tip electrode 602 has a diameter, d1, and a length L1, and first intermediate electrode 608 has a diameter, d2, and a length L2. Here, d2 is greater than or equal to d1, and/or L2 is greater than or equal to L1, such that first intermediate electrode 608 (including second sub-electrodes 614) has a surface area (A2) that is greater than or equal to a surface area (A1) of tip electrode 602 (including first sub-electrodes 612). In some embodiments, the diameter, d1, is measured in a direction perpendicular to the longitudinal axis 622 and the length, L1, is measured in a direction parallel to the longitudinal axis 622, although the diameter, d1, and the length, L1, may be measured in any orientation relative to each other and/or the longitudinal axis 622 without departing from the scope of the disclosure.

When using tip electrode 602 as the active electrode and first intermediate electrode 608 as the return electrode, first intermediate electrode 608 has a lower resistance and a lower current density than a smaller return electrode would. Accordingly, first intermediate electrode 608 generates lower electric fields (and electric field gradients) and reduces the possibility of generating a shadow lesion at first intermediate electrode 608 (as compared to a smaller return electrode). Further, first intermediate electrode 608 experiences less of a temperature rise (as compared to a smaller return electrode), and reduces the possibility of generating char, blood coagulation, and hemolysis at first intermediate electrode 608 (as compared to a smaller return electrode).

In embodiments using arrays of sub-electrodes (e.g., catheter assemblies 500 and 600), contact sensing algorithms may be used to detect which sub-electrodes are in contact with tissue. For example, tissue has a higher impedance than a blood pool. Accordingly, by determining an impedance at a sub-electrode (e.g., based on monitored voltages and/or currents at the sub-electrode), it can be determined whether or not that sub-electrode is in contact with tissue. Based on this, PFA energy may be selectively applied only to sub-electrodes that are determined to be in contact with tissue. This facilitates efficient PFA ablation using less energy, and with reduced microbubble formation, hemolysis, and blood coagulation. Further, this technique may also reduce heating in some embodiments.

In some embodiments, one or more electrodes may benefit from active cooling using irrigation. Further, in some embodiments, different electrodes may be actively cooled using different irrigants.

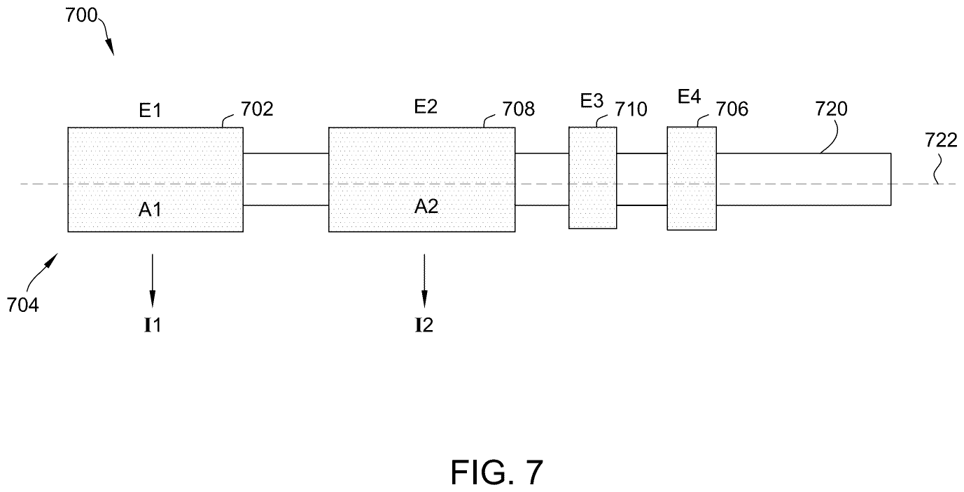

For example, FIG. 7 is a schematic diagram of another embodiment of a catheter assembly 700 that may be used with catheter 14 in system 10. Catheter assembly 700 includes a tip electrode 702 (E1) at a distal end 704 of catheter assembly 700. Catheter assembly 700 further includes a proximal electrode 706 (E4), a first intermediate electrode 708 (E2), and a second intermediate electrode 710 (E3). Tip electrode 702, proximal electrode 706, first intermediate electrode 708, and second intermediate electrode 710 are disposed along a shaft 720 that extends along a longitudinal axis 722.

Tip electrode 702 may be, for example, a dome-shaped (or sphere-shaped) electrode. Using a dome-shaped electrode as tip electrode 602 facilitates reducing edge effects, reducing the likelihood of blood coagulation and/or hemolysis. In general, smaller electrodes and/or sharper electrode edges may facilitate relatively high current density and electric field generation, possibly increasing the likelihood of heating, blood coagulation, and/or hemolysis. Further, proximal electrode 706, first intermediate electrode 708, and second intermediate electrode 710 may be, for example, ring electrodes. Alternatively, tip electrode 702, proximal electrode 706, first intermediate electrode 708, and second intermediate electrode 710 may each be any suitable type and/or shape of electrode.

In this embodiment, first intermediate electrode 708 has a surface area (A2) that is greater than or equal to a surface area (A1) of tip electrode 702. Further, in this embodiment, as shown in FIG. 7, tip electrode 702 is irrigated with a first irrigant (11), and first intermediate electrode 708 is irrigated with a second irrigant (12).

Using different irrigants facilitates shaping the electric field generated by catheter assembly 700 (and thus, controlling lesion depth). Notably, electric field strength is generally higher in lower conductivity media. Thus, in one example, I1 is a lower conductivity saline than I2. This results in a higher electric field at tip electrode 702 (i.e., at the intended lesion site) than at first intermediate electrode 708.

In some embodiments, irrigants may include cytotoxic agents (e.g., CaCl₂) to facilitate cell death. For example, I1 may include a predetermined concentration of CaCl₂ to facilitate cell death.

Although only tip electrode 702 and first intermediate electrode 708 are shown as being irrigated in FIG. 7, those of skill in the art will appreciate that proximal electrode 706 and/or second intermediate electrode 710 may be irrigated. Further, electrodes in the other embodiments described herein (e.g., catheter assemblies 300, 400, 500, and 600) may be similarly irrigated.

A method of assembling an electroporation catheter includes positioning an active electrode on a shaft that extends along a longitudinal axis and positioning a return electrode on the shaft. The active electrode has a first surface area and the return electrode has a second surface area that is greater than or equal to the first surface area to facilitate suppressing electric fields and/or preventing temperature rise at the return electrode when applying electrical energy between the active electrode and the return electrode to form a lesion proximate the active electrode. In some embodiments, the active electrode has a first length and the return electrode has a second length that is greater than or equal to the first length. The first length of the active electrode may extend in a direction parallel to the longitudinal axis and the length of the return electrode extends in a direction parallel to the longitudinal axis. In some embodiments, the active electrode has a first diameter and the return electrode has a second diameter that is greater than or equal to the first diameter. The first diameter of the active electrode and the second electrode of the return electrode may extend in a direction perpendicular to the longitudinal axis. In one non-limiting embodiment, at least one of the active electrode and the return electrode includes a plurality of independently activatable sub-electrodes.

Those of skill in the art will appreciate that the irrigation functionality described herein may be achieved using any suitable irrigation architecture. For example, the irrigation functionality may be implemented within a catheter with a flexible tip or a catheter with a relatively rigid tip. In some embodiments, separate irrigation lines are connected to different electrodes, which facilitates independent control of irrigant flow to each electrode (e.g., active and return electrodes). This also enables using different irrigants for different electrodes. Further, this allows for providing limited irrigation to return electrodes when those electrodes are not in use (e.g., during an RF ablation application). Alternatively, each electrode may include a separate irrigation port off of a shared irrigation line.

In some embodiments, at least some of the electrodes in the catheter assemblies disclosed herein (e.g., the return electrodes) may use relatively low resistance electrode materials and/or be connected to relatively low resistance electrodes wires. This facilitates increasing current to an active electrode (e.g., a tip electrode) to improve lesion formation. For example, copper may be used for the electrodes and/or electrode wires. As another example, in some embodiments, the electrodes may undergo processing (e.g., surface roughening using a laser or mechanical tool) to increase surface area and lower resistance. As another example, one or more coatings may be applied to the electrodes (e.g., irridium oxide or titanium nitride). These coatings may increase surface area (increasing capacitive behavior) and/or induce a chemical reaction effect (e.g., Faradaic charge injection that improves electron flow) to lower resistance. The increased surface area may also facilitate a reduced likelihood of blood coagulation and/or hemolysis.

The systems and methods described herein are directed to electroporation applications. An electroporation catheter includes an active electrode having a first surface area, and a return electrode having a second surface area, wherein the second surface area is greater than or equal to the first surface area to facilitate suppressing electric fields and/or preventing temperature rise at the return electrode when applying electrical energy between the active electrode and the return electrode to form a lesion proximate the active electrode.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

The invention is defined in the appended claims. A non-exhaustive list of aspects of the invention set out in the following numbered clauses is useful for understanding the invention:
1. An electroporation catheter comprising:
an active electrode having a first surface area; and
a return electrode having a second surface area, wherein the second surface area is greater than or equal to the first surface area to facilitate suppressing electric fields and/or preventing temperature rise at the return electrode when applying electrical energy between the active electrode and the return electrode to form a lesion proximate the active electrode.
2. The electroporation catheter according to clause 1, wherein the active electrode comprises a plurality of active electrodes, wherein the return electrode comprises a plurality of return electrodes, wherein a sum of the second surface areas of the plurality of return electrodes is greater than a sum of the first surface areas of the plurality of active electrodes.
3. The electroporation catheter according to clause 1 or 2, wherein the electroporation catheter is a linear catheter comprising a shaft extending along a longitudinal axis, and wherein the active electrode and the return electrode are disposed on the shaft.
4. The electroporation catheter according to any of clauses 1 to 3, wherein the active electrode comprises a tip electrode located at a distal end of the shaft, and wherein the return electrode is proximal of the tip electrode.
5. The electroporation catheter according to any of clauses 1 to 3, wherein the active electrode has a first length, wherein the return electrode has a second length, and wherein the second length is greater than or equal to the first length.
6. The electroporation catheter according to any of clauses 1 to 3, wherein the active electrode has a first length in a direction parallel to the longitudinal axis, wherein the return electrode has a second length in the direction parallel to the longitudinal axis, and wherein the second length is greater than or equal to the first length.
7. The electroporation catheter according to any of clauses 1 to 3, wherein the active electrode has a first diameter, wherein the return electrode has a second diameter, and wherein the second diameter is greater than or equal to the first diameter.
8. The electroporation catheter according to any of clauses 1 to 3, wherein the active electrode has a first diameter in a direction perpendicular to the longitudinal axis, wherein the return electrode has a second diameter in the direction perpendicular to the longitudinal axis, and wherein the second diameter is greater than or equal to the first diameter.
9. The electroporation catheter according to any preceding clause, wherein at least one of the active electrode and the return electrode comprises a plurality of independently activatable sub-electrodes.
10. The electroporation catheter according to clause 9, wherein each of the plurality of sub-electrodes comprises a flex circuit.
11. The electroporation catheter according to any preceding clause, further comprising an irrigation architecture configured to:
deliver a first irrigant to the active electrode; and
deliver a second irrigant to the return electrode.
12. The electroporation catheter according to clause 11, wherein the first irrigant has a lower conductivity than the second irrigant.
13. The electroporation catheter according to clause 11, wherein the first irrigant includes a cytotoxic agent.
14. The electroporation catheter according to any preceding clause, wherein at least one of the active electrode and the return electrode comprises one of a conductive balloon and a conductive mesh.
15. An electroporation system comprising:
a pulse generator; and
an electroporation catheter coupled to the pulse generator, the electroporation catheter comprising:
   an active electrode having a first surface area; and
   a return electrode having a second surface area, wherein the second surface area is greater than or equal to the first surface area to facilitate suppressing electric fields and/or preventing temperature rise at the return electrode when applying, using the pulse generator, electrical energy between the active electrode and the return electrode to form a lesion proximate the active electrode.
16. The electroporation system according to clause 15, wherein the active electrode comprises a plurality of active electrodes, wherein the return electrode comprises a plurality of return electrodes, wherein a sum of the second surface areas of the plurality of return electrodes is greater than a sum of the first surface areas of the plurality of active electrodes
17. The electroporation system according to clause 15 or 16, wherein the electroporation catheter is a linear catheter comprising a shaft extending along a longitudinal axis, and wherein the active electrode and the return electrode are disposed on the shaft.
18. The electroporation system according to clause 15 or 16, wherein the active electrode comprises a tip electrode located at a distal end of the shaft, and wherein the return electrode is proximal of the tip electrode.
19. The electroporation system according to any of clauses 15 to 18, wherein at least one of the active electrode and the return electrode comprises a plurality of independently activatable sub-electrodes.
20. A method of assembling an electroporation catheter, the method comprising:
positioning an active electrode on a shaft that extends along a longitudinal axis, the active electrode having a first surface area; and
positioning a return electrode on the shaft, the second electrode having a second surface area that is greater than or equal to the first surface area to facilitate suppressing electric fields and/or preventing temperature rise at the return electrode when applying electrical energy between the active electrode and the return electrode to form a lesion proximate the active electrode.
21. The method according to clause 20, wherein the active electrode has a first length, wherein the return electrode has a second length, and wherein the second length is greater than or equal to the first length.
22. The method according to clause 20, wherein the active electrode has a first length in a direction parallel to the longitudinal axis, wherein the return electrode has a second length in the direction parallel to the longitudinal axis, and wherein the second length is greater than or equal to the first length.
23. The method according to any of clauses 20 to 22, wherein the active electrode has a first diameter, wherein the return electrode has a second diameter, and wherein the second diameter is greater than or equal to the first diameter.
24. The method according to any of clauses 20 to 22, wherein the active electrode has a first diameter in a direction perpendicular to the longitudinal axis, wherein the return electrode has a second diameter in the direction perpendicular to the longitudinal axis, and wherein the second diameter is greater than or equal to the first diameter.
25. The method according to any of clauses 20 to 24, wherein at least one of the active electrode and the return electrode comprises a plurality of independently activatable sub-electrodes.

What is claimed is:

1. An electroporation catheter comprising:
an active electrode having a first surface area; and
a return electrode having a second surface area, wherein the second surface area is greater than or equal to the first surface area to facilitate suppressing electric fields and/or preventing temperature rise at the return electrode when applying electrical energy between the active electrode and the return electrode to form a lesion proximate the active electrode.

2. The electroporation catheter according to claim 1, wherein the active electrode comprises a plurality of active electrodes, wherein the return electrode comprises a plurality of return electrodes, and wherein a sum of the second surface areas of the plurality of return electrodes is greater than a sum of the first surface areas of the plurality of active electrodes.

3. The electroporation catheter according to claim 1, wherein the electroporation catheter is a linear catheter comprising a shaft extending along a longitudinal axis, and wherein the active electrode and the return electrode are disposed on the shaft.

4. The electroporation catheter according to claim 3, wherein the active electrode comprises a tip electrode located at a distal end of the shaft, and wherein the return electrode is proximal of the tip electrode.

5. The electroporation catheter according to claim 3, wherein the active electrode has a first length, wherein the return electrode has a second length, and wherein the second length is greater than or equal to the first length.

6. The electroporation catheter according to claim 3, wherein the active electrode has a first diameter, wherein the return electrode has a second diameter, and wherein the second diameter is greater than or equal to the first diameter.

7. The electroporation catheter according to claim 1, wherein at least one of the active electrodes and the return electrode comprise a plurality of independently activatable sub-electrodes.

8. The electroporation catheter according to claim 7, wherein each of the plurality of sub-electrodes comprises a flex circuit.

9. The electroporation catheter according to claim 1, further comprising an irrigation architecture configured to:
deliver a first irrigant to the active electrode; and
deliver a second irrigant to the return electrode.

10. The electroporation catheter according to claim 9, wherein the first irrigant has a lower conductivity than the second irrigant.

11. The electroporation catheter according to claim 9, wherein the first irrigant includes a cytotoxic agent.

12. The electroporation catheter according to claim 1, wherein at least one of the active electrode and the return electrode comprises one of a conductive balloon and a conductive mesh.

13. An electroporation system comprising:
a pulse generator; and
an electroporation catheter coupled to the pulse generator, the electroporation catheter comprising:
an active electrode having a first surface area; and
a return electrode having a second surface area, wherein the second surface area is greater than or equal to the first surface area to facilitate suppressing electric fields and/or preventing temperature rise at the return electrode when applying, using the pulse generator, electrical energy between the active electrode and the return electrode to form a lesion proximate the active electrode.

14. The electroporation system according to claim 13, wherein the active electrode comprises a plurality of active electrodes, wherein the return electrode comprises a plurality of return electrodes, and wherein a sum of the second surface areas of the plurality of return electrodes is greater than a sum of the first surface areas of the plurality of active electrodes.

15. The electroporation system according to claim 13, wherein the electroporation catheter is a linear catheter comprising a shaft extending along a longitudinal axis, and wherein the active electrode and the return electrode are disposed on the shaft.

16. The electroporation system according to claim 15, wherein the active electrode comprises a tip electrode located at a distal end of the shaft, and wherein the return electrode is proximal of the tip electrode.

17. The electroporation system according to claim 13, wherein at least one of the active electrode and the return electrode comprises a plurality of independently activatable sub-electrodes.

18. A method of assembling an electroporation catheter, the method comprising:
positioning an active electrode on a shaft that extends along a longitudinal axis, the active electrode having a first surface area; and
positioning a return electrode on the shaft, the return electrode having a second surface area that is greater than or equal to the first surface area to facilitate suppressing electric fields and/or preventing temperature rise at the return electrode when applying electrical energy between the active electrode and the return electrode to form a lesion proximate the active electrode.

19. The method according to claim 18, wherein the active electrode has a first length, wherein the return electrode has a second length, and wherein the second length is greater than or equal to the first length.

20. The method according to claim 18, wherein the active electrode has a first diameter, wherein the return electrode has a second diameter, and wherein the second diameter is greater than or equal to the first diameter.
